(19) United States
(12) Patent Application Publication
(10) Pub. No.: US 2024/0399347 A1
(43) Pub. Date: Dec. 5, 2024

(54) CATALYST FOR CUMENE HYDROPEROXIDE DECOMPOSITION AND PROCESS FOR PREPARATION THEREOF

(71) Applicant: PTT GLOBAL CHEMICAL PUBLIC COMPANY LIMITED, Bangkok (TH)

(72) Inventors: Suchada TANG-AMORNSUKSAN, Bangkok (TH); Sitthiphong PENGPANICH, Bangkok (TH); Papapida PORNSURIYASAK, Bangkok (TH)

(21) Appl. No.: 18/724,157

(22) PCT Filed: Dec. 28, 2022
(86) PCT No.: PCT/IB2022/062829

§ 371 Date: Jun. 25, 2024

(30) Foreign Application Priority Data
Dec. 30, 2021 (TH) 2101008264

(51) Int. Cl. B01J 29/70; B01J 37/06; B01J 37/08; B01J 37/12; C01B 39/02; C07C 37/08

(52) U.S. Cl. CPC B01J 29/7007; B01J 37/06; B01J 37/08; B01J 37/12; C01B 39/026; C07C 37/08; B01J 2229/37; C07C 2529/70

(57) ABSTRACT

The present invention relates to a catalyst for cumene hydroperoxide decomposition, wherein said catalyst has good efficacy, provides good decomposition of cumene hydroperoxide and good selectivity to main products, provides high yield percentage of alpha methyl styrene, and reduces the formation of unwanted byproducts. Said catalyst comprises zeolite beta, wherein said catalyst has the following characteristics: a) the mole ratio of silica to alumina is from 30 to 100; and b) the peak area ratio obtained from the analysis using ²⁷Al magic angle spinning-nuclear magnetic resonance (²⁷Al MAS-NMR) technique according to the following relation: (A+C+D)/B is from 0.1 to 0.3; wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis with Lorentz/Gauss function under the condition that the minimum distance between peaks for independent integration (AZFW) is equal to 0.5 ppm. Moreover, this invention relates to a preparation process of the catalyst comprising the step of contacting zeolite beta with polycarboxylic acid solution and calcination at the temperature in the range from 400 to 700° C., wherein said polycarboxylic acid has the pKa of the first dissociation from 1.5 to 3.5 and has the molecular structure with at least one side having size larger than 6 Å. This invention also relates to a process for cumene hydroperoxide decomposition using said catalyst or catalyst prepared from said preparation process of the catalyst.

### TECHNICAL FIELD

The present invention relates to the field of chemistry, in particular, to the catalyst for cumene hydroperoxide decomposition, process for preparing said catalyst, and process for cumene hydroperoxide decomposition using said catalyst.

### BACKGROUND OF THE INVENTION

Most of the industrial productions of phenol start from cumene. Said phenol production process comprises the following main steps: (1) oxidation of cumene; (2) concentration of the mixture obtained from oxidation step by removing cumene; (3) decomposition using acid; and (4) neutralization. From said steps above. the oxidation process of cumene generally occurs incomplete oxidation. This gives products from cumene oxidation comprising cumene hydroperoxide (CHP) and dimethyl phenylcarbinol (DMPC) in which dimethyl phenylcarbinol is the byproduct obtained from incomplete oxidation of cumene. When both of cumene hydroperoxide and dimethyl phenylcarbinol are subjected to decomposition step using acid, cumene hydroperoxide will be decomposed to the products that are phenol and acetone. Whereas dimethyl phenylcarbinol can occur dehydration to the product that is alpha-methyl styrene (AMS) or can react with cumene hydroperoxide to yield the product that is dicumyl peroxide (DCP) which can further decompose into alpha-methyl styrene, phenol, and acetone. The alpha-methyl styrene formed will be subjected to hydrogenation process to convert to cumene and then subject the obtained cumene to the initial process for reusing. Therefore, there are 3 main valuable products from cumene hydroperoxide decomposition process which are phenol, acetone, and alpha-methyl styrene. However, alpha-methyl styrene obtained from reaction of dimethyl phenylcarbinol always losses into the unwanted byproduct which are alpha-methyl styrene dimers (AMS dimers) and cumylphenol formed from the reaction between alpha-methyl styrene and phenol under acidic condition. This means that it causes the loss of alpha-methyl styrene, which is the main product and is valuable for producing the cumene reactant for reusing in the process of phenol production, and also the loss of main product of process that is phenol.

The process of cumene hydroperoxide decomposition can be operated by using acid solution as the catalyst, in which the acid solution commonly used is sulfuric acid. Nevertheless, the use of said acid solution catalyst still has unwanted properties such as the need of neutralization step, increase in waste, corrosion to the equipment, low specificity for the formation of desired product, and difficulty and complexity of phenol purification process. Therefore, there is the continuous studies and reports on the use of solid catalyst for the process of cumene hydroperoxide decomposition instead of the use of acid solution as the catalyst with the motivation in lower cost and lower formation of unwanted byproducts. The documents that disclose about the use of solid catalyst for the cumene hydroperoxide decomposition process are as follows.

Patent documents U.S. Pat. Nos. 4,490,565A and 4,490,566A disclose the use of solid catalysts that were zeolite beta and ZSM-5 zeolite in the cumene hydroperoxide decomposition process, respectively. Said process was operated at quite high temperature from 80 to 200° C., preferably 80 to 120° C. From the patent document U.S. Pat. No. 4,490,565A, it showed good conversion percentage of cumene hydroperoxide at low temperature for zeolite beta comparing to ZSM-5 zeolite disclosed in patent document U.S. Pat. No. 4,490,566A.

Patent documents EP0 4 9 2 8 0 7 A2 discloses the use of solid catalysts that were mordonite-type zeolite or faujasite-type zeolite selected from Y-zeolite, ammonium exchanged Y-zeolite, thermally-stabilized Y-zeolite, dealuminated Y-zeolite, rare-earth exchanged Y-zeolite, and fluoride-treated Y-zeolite for the cumene hydroperoxide decomposition process. The experimental results showed that Y-zeolite treated with ammonium fluoride gave better conversion percentage of cumene hydroperoxide but gave product composition comprising alpha-methyl styrene with lower proportion and cumyl phenol, which was the unwanted byproduct, with higher proportion compared to untreated Y-zeolite. However, said document does not disclose the result of treatment on zeolite beta catalyst.

Patent documents U.S. Pat. No. 6,441,251B1 discloses the process for producing phenol and acetone from cumene hydroperoxide. Said process comprised the step of contacting cumene hydroperoxide with solid-acid catalyst that was silicate or aluminosilicate, wherein said catalyst comprised sulfonate functionality. The sulfonic acid group would enhance the activity of catalyst for cumene hydroperoxide decomposition. For example, MCM-41 zeolite catalyst containing sulfonic acid functionality had higher activity than MCM-41 zeolite catalyst without sulfonic acid functionality. Moreover, when considering the conversion of dimethyl phenylcarbinol, it was found that MCM-41 zeolite catalyst containing sulfonic acid functionality gave clearly better conversion of dimethyl phenylcarbinol than MCM-41 zeolite catalyst without sulfonic acid functionality.

Patent documents U.S. Pat. No. 7,888,537B2 discloses the use of layered and non-layered solid catalyst for cumene hydroperoxide decomposition process. The layered solid catalyst comprised an inner core selected from refractory inorganic oxide, silicon carbide, and metal. The outer layer was an acidic material such as zeolite beta. This document discloses the belief that the use of said layered catalyst would reduce the formation of unwanted byproducts. However, the preparation process of layered catalyst was complicated and the thickness of obtained layer would affect the formation of alpha-methyl styrene differently which depended on the material of inner core.

From the above reasons, this invention aims to prepare the catalyst for cumene hydroperoxide decomposition, process for preparing said catalyst, and process for cumene hydroperoxide decomposition using said catalyst. Said catalyst has good efficacy, provides good decomposition of cumene hydroperoxide, provides good selectivity to main products and high yield percentage of alpha methyl styrene, and reduces the formation of unwanted byproducts. Also, said catalyst can be prepared from simple and uncomplicated process.

### SUMMARY OF THE INVENTION

The present invention aims to prepare a catalyst for cumene hydroperoxide decomposition, wherein said catalyst has good efficacy, provides good decomposition of cumene hydroperoxide, provides good selectivity to main products and high yield percentage of alpha methyl styrene, and reduces the formation of unwanted byproducts. Also, said catalyst can be prepared from simple and uncomplicated process. Said catalyst comprises zeolite beta, wherein said catalyst has the following characteristics:
a) the mole ratio of silica to alumina is from 30 to 100; and
b) the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

In one aspect of the invention, this invention relates to a preparation process of the catalyst for cumene hydroperoxide decomposition comprising the step of contacting zeolite beta with polycarboxylic acid solution and calcination at the temperature in the range from 400 to 700° C., wherein said polycarboxylic acid has the pKa of the first dissociation from 1.5 to 3.5 and has the molecular structure with at least one side having size larger than 6 Å.

In another aspect of the invention, this invention also relates to a process for cumene hydroperoxide decomposition comprising the contact of the feed line comprising cumene hydroperoxide to the catalyst comprising zeolite beta in the reactor, and the separation of phenol and acetone from the product line, wherein said catalyst has the following characteristics:
a) the mole ratio of silica to alumina is from 30 to 100; and
b) the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

### DETAILED DESCRIPTION

The present invention relates to the catalyst for cumene hydroperoxide decomposition, process for preparing said catalyst, and process for cumene hydroperoxide decomposition using said catalyst. Said catalyst has good efficacy, provides good decomposition of cumene hydroperoxide, provides good selectivity to main products and high yield percentage of alpha methyl styrene, and reduces the formation of unwanted byproducts. Also, said catalyst can be prepared from simple and uncomplicated process, which will be describe in the following aspects of the invention.

Any aspect being described herein also means to include the application to other aspects of this invention unless stated otherwise.

### Definitions

Technical terms or scientific terms used herein have definitions as understood by an ordinary person skilled in the art unless stated otherwise.

Any tools, equipment, methods, or chemicals named herein mean tools, equipment, methods, or chemicals being operated or used commonly by those person skilled in the art unless stated otherwise that they are tools, equipment, methods, or chemicals specific only in this invention.

Use of singular noun or singular pronoun with “comprising” in claims or specification means “one” and also including “one or more”, “at least one”, and “one or more than one”.

All compositions and/or methods disclosed and claims in this application are intended to cover embodiments from any operation, performance, modification, or adjustment any factors without any experiment that significantly different from this invention, and obtain with object with utility and resulted as same as the present embodiment according to person ordinary skilled in the art although without specifically stated in claims. Therefore, substitutable or similar object to the present embodiment, including any minor modification or adjustment that can be apparent to person skilled in the art should be construed as remains in spirit, scope, and concept of invention as appeared in appended claims.

Throughout this application, term “about” means any number that appeared or expressed herein that could be varied or deviated from any error of equipment, method, or personal using said equipment or method, including variations or deviations occurred from changes in reaction conditions of uncontrollable factors such as humidity and temperature.

The zeolite beta (BEA) in this invention means microporous alumino-silicate compound comprising silicon, aluminium, and oxygen in the structure and having three-dimensional structure which connects and arranges to the crystalline framework comprising three intersecting 12-ring channels. The zeolite beta may be commercial zeolite, natural zeolite, or zeolite prepared by any method.

Polycarboxylic acid in this invention means the acid comprising at least 2 carboxylic groups. The polycarboxylic acid may comprise other element atoms which are the atom of element that is not carbon, hydrogen, and oxygen. Said other element atoms include but not limited to nitrogen, phosphorus, sulfur, etc.

The size of molecular structure in this invention means the size of any side or at least one side of the molecule in three-dimension form. Said size of molecular structure may be the size of molecular structure that has been commonly disclosed or may be the size of molecular structure that can be calculated by any method such as computer program, etc. The term “size of molecular structure” means including molecular size, molecular dimension, molecular length, and molecular diameter.

The term “the molecular structure with at least one side” means “at least one side of the molecular structure”, and also means “any side of the molecular structure”, “one side of the molecular structure”, and “one side or more of the molecular structure”.

The pKa in this invention means the pKa of the first dissociation. Said pKa may be the pKa that has been commonly disclosed, or may be calculated by any method such as titration method, etc.

The peak deconvolution analysis with Lorentz/Gauss function in this invention means the peak deconvolution analysis using equation of Lorentz function together with equation of Gauss function.

Hereafter, invention embodiments are shown without any purpose to limit any scope of the invention.

This invention relates to the catalyst for cumene hydroperoxide decomposition, wherein said catalyst has good efficacy, provides good decomposition of cumene hydroperoxide, provides good selectivity to main products and high yield percentage of alpha methyl styrene, and reduces the formation of unwanted byproducts. Also, said catalyst can be prepared from simple and uncomplicated process. Said catalyst comprises zeolite beta, wherein said catalyst has the following characteristics:
a) the mole ratio of silica to alumina is from 30 to 100; and
b) the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

In one aspect of the invention, the mole ratio of silica to alumina is from 50 to 100, preferably from 50 to 80.

In one aspect of the invention, the peak area ratio obtained from the analysis using ²⁷Al magic angle spinning-nuclear magnetic resonance technique according to the following relation: (A+C+D)/B is from 0.1 to 0.25.

In one aspect of the invention, said catalyst is prepared from the process comprising the step of contacting zeolite beta with polycarboxylic acid solution and calcination at the temperature in the range from 400 to 700° C., wherein said polycarboxylic acid has the pKa of the first dissociation from 1.5 to 3.5 and has the molecular structure with at least one side having size larger than 6 Å.

In one aspect of the invention, zeolite beta prior to contacting with polycarboxylic acid solution has the mole ratio of silica to alumina in the range from 20 to 50, preferably the mole ratio of silica to alumina in the range from 25 to 40.

In one aspect of the invention, zeolite beta prior to contacting with polycarboxylic acid solution has the surface area in the range from 300 to 800 m²/g.

In one aspect of the invention, zeolite beta prior to contacting with polycarboxylic acid solution has the pore size in the range from 6 to 9 Å.

In one aspect of the invention, said polycarboxylic acid has the pKa of the first dissociation in the range from 2.5 to 3.0.

In one aspect of the invention, said polycarboxylic acid has the molecular structure with at least one side having size larger than 6 Å but not more than 30 Å. Preferably, said polycarboxylic acid has the molecular structure with at least one side having size from 8 to 20 Å.

In one aspect of the invention, polycarboxylic acid is selected from, but not limited to tartaric acid, citric acid, isocitric acid, aconitric acid, citraconic acid, 1,2,3,4-butanetetracarboxylic acid, ethylenediaminetetraacetic acid, nitriloacetic acid, diethylenetriaminepentaacetic acid (pentetic acid), hydroxyethylethylenediaminetriacetic acid, ethylenediaminedisuccinic acid, iminodiacetic acid, iminodisuccinic acid, methylglycinediacetic acid, 2-butenedioic acid, 1,1′-[[(3-carboxy-1-oxo-2-propen-1-yl)imino]di-2,1-ethanediyl] ester (DHEA), or mixture thereof.

In one aspect of the invention, the weight ratio of zeolite beta to polycarboxylic acid solution is in the range from 1:10 to 1:30, preferably in the range from 1:15 to 1:25, most preferably about 1:20.

In one aspect of the invention, the ratio of polycarboxylic acid to zeolite beta is in the range from 1 to 6 moles of polycarboxylic acid per kg of zeolite beta, preferably in the range from 1 to 4 moles of polycarboxylic acid per kg of zeolite beta.

In one aspect of the invention, contacting zeolite beta with polycarboxylic acid solution is performed at the temperature in the range from 50 to 90° C., preferably at the temperature in the range from 60 to 80° C., most preferably at the temperature in the range from 65 to 75° C.

In one aspect of the invention, the time for contacting zeolite beta with polycarboxylic acid solution is from 1 to 5 hours, preferably from 2 to 4 hours.

In one aspect of the invention, said process may further comprise the step of washing with solvent and drying.

In one aspect of the invention, said process further comprising the step of washing with solvent and drying, in which the drying is performed at the temperature in the range from 60 to 120° C. Preferably, the drying is performed at the temperature in the range from 70 to 100° C.

In one aspect of the invention, said process further comprising the step of washing with solvent and drying, in which the drying may be performed by, but not limited to conventional drying using oven, drying via natural evaporation, stirred evaporation, vacuum drying, drying by rotary evaporator, etc.

In one aspect of the invention, the calcination is performed at the temperature in the range from 500 to 600° C.

In one aspect of the invention, the time for calcination is from 3 to 6 hours.

In one aspect of the invention, the catalyst described above is used for cumene hydroperoxide decomposition.

In one aspect of the invention, the catalyst described above is used for cumene hydroperoxide decomposition process further comprising the dehydration of dimethyl phenylcarbinol.

In another aspect of the invention, this invention relates to the preparation process of the catalyst for cumene hydroperoxide decomposition comprising the step of contacting zeolite beta with the polycarboxylic acid solution, and calcination at the temperature in the range from 400 to 700° C., wherein said polycarboxylic acid has the pKa of the first dissociation from 1.5 to 3.5 and has the molecular structure with at least one side having size larger than 6 Å.

In one aspect of the invention, said catalyst has the peak area ratio obtained from the analysis using ²⁷Al magic angle spinning-nuclear magnetic resonance (²⁷Al MAS-NMR) technique according to the following relation: (A+C+D)/B is from 0.1 to 0.3, wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm. Most preferably, (A+C+D)/B is from 0.1 to 0.25.

In one aspect of the invention, said catalyst has the mole ratio of silica to alumina from 30 to 100, preferably from 50 to 100, most preferably from 50 to 80.

In one aspect of the invention, zeolite beta prior to contacting with polycarboxylic acid solution has the mole ratio of silica to alumina in the range from 20 to 50, preferably the mole ratio of silica to alumina in the range from 25 to 40.

In one aspect of the invention, zeolite beta prior to contacting with polycarboxylic acid solution has the surface area in the range from 300 to 800 m²/g.

In one aspect of the invention, zeolite beta prior to contacting with polycarboxylic acid solution has the pore size in the range from 6 to 9 Å.

In one aspect of the invention, said polycarboxylic acid has the pKa of the first dissociation in the range from 2.5 to 3.0.

In one aspect of the invention, said polycarboxylic acid has the molecular structure with at least one side having size larger than 6 Å but not more than 30 Å. Preferably, said polycarboxylic acid has the molecular structure with at least one side having size from 8 to 20 Å.

In one aspect of the invention, polycarboxylic acid is selected from, but not limited to tartaric acid, citric acid, isocitric acid, aconitric acid, citraconic acid, 1,2,3,4-butanetetracarboxylic acid, ethylenediaminetetraacetic acid, nitriloacetic acid, diethylenetriaminepentaacetic acid (pentetic acid), hydroxyethylethylenediaminetriacetic acid, ethylenediaminedisuccinic acid, iminodiacetic acid, iminodisuccinic acid, methylglycinediacetic acid, 2-butenedioic acid, 1,1′-[[(3-carboxy-1-oxo-2-propen-1-yl)imino]di-2,1-ethanediyl] ester (DHEA), or mixture thereof.

In one aspect of the invention, the weight ratio of zeolite beta to polycarboxylic acid solution is in the range from 1:10 to 1:30, preferably in the range from 1:15 to 1:25, most preferably about 1:20.

In one aspect of the invention, the ratio of polycarboxylic acid to zeolite beta is in the range from 1 to 6 moles of polycarboxylic acid per kg of zeolite beta, preferably in the range from 1 to 4 moles of polycarboxylic acid per kg of zeolite beta.

In one aspect of the invention, contacting zeolite beta with polycarboxylic acid solution is performed at the temperature in the range from 50 to 90° C., preferably at the temperature in the range from 60 to 80° C., most preferably at the temperature in the range from 65 to 75° C.

In one aspect of the invention, the time for contacting zeolite beta with polycarboxylic acid solution is from 1 to 5 hours, preferably from 2 to 4 hours.

In one aspect of the invention, said process may further comprise the step of washing with solvent and drying.

In one aspect of the invention, said process further comprising the step of washing with solvent and drying, in which the drying is performed at the temperature in the range from 60 to 120° C., preferably from 70 to 100° C.

In one aspect of the invention, said process further comprising the step of washing with solvent and drying, in which the drying may be performed by, but not limited to conventional drying using oven, drying via natural evaporation, stirred evaporation, vacuum drying, drying by rotary evaporator, etc.

In one aspect of the invention, the calcination is performed at the temperature in the range from 500 to 600° C.

In one aspect of the invention, the time for calcination is from 3 to 6 hours.

In another aspect of the invention, this invention relates to the process for cumene hydroperoxide decomposition comprising the contact of the feed line comprising cumene hydroperoxide to the catalyst described above or the catalyst prepared from the preparation process of the catalyst as described above in the reactor, and the separation of phenol and acetone from the product line.

In one aspect of the invention, the contact of the feed line comprising cumene hydroperoxide to the catalyst is performed at the temperature in the range from 50 to 100° C., preferably at the temperature in the range from 55 to 70° C., most preferably at the temperature in the range from 55 to 65° C.

In one aspect of the invention, the feed line comprising cumene hydroperoxide may further comprise dimethyl phenylcarbinol.

In one aspect of the invention, the process for cumene hydroperoxide decomposition, in which the feed line comprises cumene hydroperoxide and further comprises dimethyl phenylcarbinol, may further comprise the dehydration of dimethyl phenylcarbinol.

In one aspect of the invention, said feed line comprising cumene hydroperoxide and further comprising dimethyl phenylcarbinol has the proportion of cumene hydroperoxide in an amount of 70 to 90% by weight and the proportion of dimethyl phenylcarbinol in an amount of 1 to 10% by weight, preferably the proportion of cumene hydroperoxide in an amount of 75 to 90% by weight and the proportion of dimethyl phenylcarbinol in an amount of 2 to 7% by weight.

In one aspect of the invention, said feed line comprising cumene hydroperoxide and further comprising dimethyl phenylcarbinol has the weight ratio of cumene hydroperoxide to dimethyl phenylcarbinol in the range from 7 to 90, preferably in the range from 10 to 36.

In one aspect of the invention, the process for cumene hydroperoxide decomposition may be performed in the reactor, but not limited to the stirred tank reactor or the fixed-bed reactor which may be performed in batch or continuous manner, or may be performed in fixed bed system, moving bed system, fluidized bed system, or batch system.

Generally, any person skilled in this art can adjust the conditions for cumene hydroperoxide decomposition to be suitable for catalyst, type and composition of the feed line, and reactor system.

The following examples are for demonstrating one aspect of this invention only, not for limiting the scope of this invention in any way.

### Preparation Process of Catalyst

In the preparation process of the catalyst for cumene hydroperoxide decomposition comprising zeolite beta, said catalyst may be prepared from the process comprising the step of contacting zeolite beta with polycarboxylic acid solution and calcination at the temperature in the range from 400 to 700° C., wherein said polycarboxylic acid had the pKa of the first dissociation from about 1.5 to 3.5 and had the molecular structure with at least one side having size larger than 6 Å.

The details of each step in the preparation process of the catalyst can be explained as follows.

Contacting Zeolite Beta with Polycarboxylic Acid Solution

The contacting zeolite beta with polycarboxylic acid solution can be performed by preparing polycarboxylic acid solution at the ratio of polycarboxylic acid to zeolite beta in the range from about 1 to 6 moles of polycarboxylic acid per kg of zeolite beta. Then, zeolite beta was added into said polycarboxylic acid solution at the weight ratio of zeolite beta to polycarboxylic acid solution in the range from about 1:10 to 1:30. After that, said mixture obtained was stirred and heated at the temperature in the range from about 50 to 90° C. for about 1 to 5 hours.

Calcination at the Temperature in the Range from 400 to 700° C.

The calcination at the temperature in the range from 400 to 700° C. can be performed by subjecting the mixture to the calcination at the temperature in the range from about 400 to 700° C. for about 1 to 5 hours with the heating rate of about 2° C./min.

Said preparation process of the catalyst may further comprise the step of washing with solvent and drying prior to the calcination. This can be performed by washing the solid with deionized water until the water obtained after washing has the same pH as the water before washing. Then, the mixture was dried by drying method at the temperature in the range from 60 to 120° C.

### Example of the Preparation of the Comparative Catalyst and the Catalyst According to the Invention

### Comparative Catalyst Sample 1

Comparative catalyst sample 1 was the catalyst comprising the commercial MCM-22 zeolite having the mole ratio of silica to alumina (SiO₂/Al₂O₃) of about 32.

### Comparative Catalyst Sample 2

Comparative catalyst sample 2 was the catalyst comprising the commercial ZSM-5 zeolite having the mole ratio of silica to alumina (SiO₂/Al₂O₃) of about 30.

### Comparative Catalyst Sample 3

Comparative catalyst sample 3 was the catalyst comprising the commercial zeolite beta having the mole ratio of silica to alumina (SiO₂/Al₂O₃) of about 30.

### Comparative Catalyst Sample 4

Comparative catalyst sample 4 was the catalyst comprising the commercial zeolite beta having the mole ratio of silica to alumina (SiO₂/Al₂O₃) of about 53.

### Comparative Catalyst Sample 5

Comparative catalyst sample 5 was the catalyst comprising the commercial zeolite beta having the mole ratio of silica to alumina (SiO₂/Al₂O₃) of about 233.

### Comparative Catalyst Sample 6

Comparative catalyst sample 6 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to formic acid at the ratio of formic acid to zeolite beta of about 2 moles of formic acid per kg of zeolite beta. The contact was performed at the weight ratio of zeolite beta to formic acid solution of about 1:20. After that, said mixture obtained was stirred and heated at the temperature about 70° C. for about 3 hours. The calcination step was performed at the temperature about 540° C. for about 4 hours.

### Comparative Catalyst Sample 7

Comparative catalyst sample 7 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to formic acid at the ratio of formic acid to zeolite beta of about 4 moles of formic acid per kg of zeolite beta. The contact was performed at the weight ratio of zeolite beta to formic acid solution of about 1:20. After that, said mixture obtained was stirred and heated at the temperature about 70° C. for about 3 hours. The calcination step was performed at the temperature about 540° C. for about 4 hours.

### Comparative Catalyst Sample 8

Comparative catalyst sample 8 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to oxalic acid at the ratio of oxalic acid to zeolite beta of about 0.2 moles of oxalic acid per kg of zeolite beta. The contact was performed at the weight ratio of zeolite beta to oxalic acid solution of about 1:20. After that, said mixture obtained was stirred and heated at the temperature about 70° C. for about 3 hours. The calcination step was performed at the temperature about 540° C. for about 4 hours.

### Comparative Catalyst Sample 9

Comparative catalyst sample 9 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to oxalic acid at the ratio of oxalic acid to zeolite beta of about 1 mole of oxalic acid per kg of zeolite beta. The contact was performed at the weight ratio of zeolite beta to oxalic acid solution of about 1:20. After that, said mixture obtained was stirred and heated at the temperature about 70° C. for about 3 hours. The calcination step was performed at the temperature about 540° C. for about 4 hours.

### Comparative Catalyst Sample 10

Comparative catalyst sample 10 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to oxalic acid at the ratio of oxalic acid to zeolite beta of about 2 moles of oxalic acid per kg of zeolite beta. The contact was performed at the weight ratio of zeolite beta to oxalic acid solution of about 1:20. After that, said mixture obtained was stirred and heated at the temperature about 70° C. for about 3 hours. The calcination step was performed at the temperature about 540° C. for about 4 hours.

### Comparative Catalyst Sample 11

Comparative catalyst sample 11 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid was changed to oxalic acid at the ratio of oxalic acid to zeolite beta of about 4 moles of oxalic acid per kg of zeolite beta. The contact was performed at the weight ratio of zeolite beta to oxalic acid solution of about 1:20. After that, said mixture obtained was stirred and heated at the temperature about 70° C. for about 3 hours. The calcination step was performed at the temperature about 540° C. for about 4 hours.

### Catalyst Sample According to the Invention 1

Catalyst sample according to the invention 1 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid being used was citric acid at the ratio of polycarboxylic acid to zeolite beta of about 1.4 moles of polycarboxylic acid per kg of zeolite beta. The contact was performed at the weight ratio of zeolite beta to polycarboxylic acid solution of about 1:20. After that, said mixture obtained was stirred and heated at the temperature about 70° C. for about 3 hours. The calcination step was performed at the temperature about 540° C. for about 4 hours.

### Catalyst Sample According to the Invention 2

Catalyst sample according to the invention 2 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid being used was citric acid at the ratio of polycarboxylic acid to zeolite beta of about 2 moles of polycarboxylic acid per kg of zeolite beta. The contact was performed at the weight ratio of zeolite beta to polycarboxylic acid solution of about 1:20. After that, said mixture obtained was stirred and heated at the temperature about 70° C. for about 3 hours. The calcination step was performed at the temperature about 540° C. for about 4 hours.

### Catalyst Sample According to the Invention 3

Catalyst sample according to the invention 3 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid being used was citric acid at the ratio of polycarboxylic acid to zeolite beta of about 4 moles of polycarboxylic acid per kg of zeolite beta. The contact was performed at the weight ratio of zeolite beta to polycarboxylic acid solution of about 1:20. After that, said mixture obtained was stirred and heated at the temperature about 70° C. for about 3 hours. The calcination step was performed at the temperature about 540° C. for about 4 hours.

### Catalyst Sample According to the Invention 4

Catalyst sample according to the invention 4 was prepared from the process described above. In the step of contacting zeolite beta with polycarboxylic acid solution, the catalyst comprising the commercial zeolite beta having the mole ratio of silica to alumina (SiO₂/Al₂O₃) of about 30 was used and the polycarboxylic acid being used was 2-butenedioic acid, 1,1′-[[(3-carboxy-1-oxo-2-propen-1-yl)imino]di-2,1-ethanediyl] ester (DHEA) at the ratio of polycarboxylic acid to zeolite beta of about 1.4 moles of polycarboxylic acid per kg of zeolite beta. The contact was performed at the weight ratio of zeolite beta to polycarboxylic acid solution of about 1:20. After that, said mixture obtained was stirred and heated at the temperature about 70° C. for about 3 hours. The calcination step was performed at the temperature about 540° C. for about 4 hours.

### Catalyst Sample According to the Invention 5

Catalyst sample according to the invention 5 was prepared from the process described above. In the step of contacting zeolite beta with polycarboxylic acid solution, the catalyst comprising the commercial zeolite beta having the mole ratio of silica to alumina (SiO₂/A12O₃) of about 30 was used and the polycarboxylic acid being used was 2-butenedioic acid, 1,1′-[[(3-carboxy-1-oxo-2-propen-1-yl)imino]di-2,1-ethanediyl] ester (DHEA) at the ratio of polycarboxylic acid to zeolite beta of about 2 moles of polycarboxylic acid per kg of zeolite beta. The contact was performed at the weight ratio of zeolite beta to polycarboxylic acid solution of about 1:20. After that, said mixture obtained was stirred and heated at the temperature about 70° C. for about 3 hours. The calcination step was performed at the temperature about 540° C. for about 4 hours.

### Testing of the Efficacy of the Catalyst in the Cumene Hydroperoxide Decomposition

The testing of the efficacy of the catalyst in the cumene hydroperoxide decomposition may be performed as follows. The cumene hydroperoxide solution was prepared in the flask at the concentration about 5% by weight of cumene hydroperoxide in acetone from about 85% cumene hydroperoxide obtained from phenol production process. Said solution prepared comprised about 0.3% by weight of dimethyl phenylcarbinol. Then, the catalyst was added into cumene hydroperoxide solution with the catalyst proportion of about 0.5% by weight of catalyst to total reactants. The reflux reaction was performed at the temperature about 60° C. along with the continuous stir for about 30 min. After the reaction was completed, the reaction was stopped by reducing the temperature to about 0 to 4° C. Then, the catalyst was separated from the mixture and the obtained mixture was further analyzed for its composition.

### Analysis of Characteristics and Structure of Zeolite

The analysis of characteristics and structure of zeolite could be performed by using x-ray diffraction (XRD) technique.

Analysis of Silica to Alumina (SiO₂/Al₂O₃) Ratio in the Catalyst

The analysis of silica to alumina (SiO₂/Al₂O₃) ratio in the catalyst could be performed by using the x-ray fluorescence (XRF) technique.

### Analysis of Characteristics of the Catalyst

The analysis of characteristics of the catalyst could be performed by using ²⁷Al magic angle spinning-nuclear magnetic resonance (²⁷Al MAS-NMR) technique. The analytical result obtained from said technique would comprise peaks at different positions, in which the determination for peak areas at different positions obtained from above technique could be performed using the peak deconvolution analysis with Lorentz/Gauss function under the condition that the minimum distance between peaks for independent integration (AZFW) was equal to 0.5 ppm.

TABLE 1
Efficacy of the catalyst comprising different zeolites in the cumene hydroperoxide
decomposition under the following conditions: the feed line comprised 10% by
weight of cumene hydroperoxide and 0.6% by weight of dimethyl phenylcarbinol
in acetone solvent; the reaction was performed at 60° C. for 1 hour; and
the proportion of the catalyst to total reactants was about 5% by weight

| | | | | | Yield of | |
|---|---|---|---|---|---|---|
| | Mole ratio of | Conversion of | | Conversion of | alpha- | Yield of |
| | silica to | cumene | Yield of | dimethyl | methyl | para-cumyl |
| | alumina | hydroperoxide | phenol | phenylcarbinol | styrene | phenol |
| Sample | (SiO₂/Al₂O₃) | (%) | (%) | (%) | (%) | (%) |
| Comparative | 32 | 99 | 82 | 69 | 20 | 0 |
| catalyst 1 | | | | | | |
| Comparative | 30 | 87 | 60 | 57 | 13 | 5.6 |
| catalyst 2 | | | | | | |
| Comparative | 30 | 100 | 94 | 98 | 93 | 0 |
| catalyst 3 | | | | | | |

From table 1, where comparing the efficacy of the catalyst in the cumene hydroperoxide decomposition using different zeolite catalysts, it was found that the comparative catalyst 3, which was the catalyst comprising zeolite beta, gave the best conversion of cumene hydroperoxide, the best conversion of dimethyl phenylcarbinol, the best yield of phenol, and the best yield of alpha-methyl styrene.

TABLE 2
Properties of different acids used in the preparation process
of catalyst for the cumene hydroperoxide decomposition

| | pKa of | Size range of |
|---|---|---|
| | the first | molecular |
| Acid | dissociation | structure ^{a} (Å) |
| Formic acid | 3.7 | 2-3 |
| Oxalic acid | 1.3 | 3-5 |
| Citric acid | 3.1 | 5-8 |
| 2-butenedioic acid, | 3.0 | 7-15 |
| 1,1′-[[(3-carboxy-1-oxo-2- | | |
| propen-1-yl)imino]di-2,1- | | |
| ethanediyl] ester (DHEA) | | |

^{a} The size range of molecular structure shown in the table was the size range of any side of molecular structure in the three-dimensional form from the lowest value to the highest value.

TABLE 3
Peak areas of the catalyst at different positions obtained from
the analysis using ²⁷Al magic angle spinning-nuclear magnetic resonance
(²⁷Al MAS-NMR) technique, wherein the peak deconvolution analysis
was analyzed with Lorentz/Gauss function under the condition
that the minimum distance between peaks for independent integration
(AZFW) was equal to 0.5 ppm

| | ^{a}Peak | ^{b}Peak | ^{c}Peak | ^{d}Peak | ^{e}Peak area ratio |
|---|---|---|---|---|---|
| Sample | area A | area B | area C | area D | (A + C + D)/B |
| Comparative | 1360 | 20589 | 5504 | 3438 | 0.50 |
| catalyst 3 | | | | | |
| Comparative | 43 | 21855 | 4811 | 5150 | 0.46 |
| catalyst 6 | | | | | |
| Comparative | 147 | 20006 | 3975 | 2713 | 0.34 |
| catalyst 7 | | | | | |
| Comparative | 0 | 15903 | 0 | 867 | 0.05 |
| catalyst 10 | | | | | |
| Comparative | 0 | 14199 | 0 | 337 | 0.02 |
| catalyst 11 | | | | | |
| Catalyst | 1994 | 17724 | 703 | 2143 | 0.27 |
| according to | | | | | |
| the invention 1 | | | | | |
| Catalyst | 158 | 17555 | 1738 | 1361 | 0.19 |
| according to | | | | | |
| the invention 2 | | | | | |
| Catalyst | 20 | 18181 | 320 | 1704 | 0.11 |
| according to | | | | | |
| the invention 4 | | | | | |
| Catalyst | 745 | 18893 | 1696 | 1574 | 0.21 |
| according to | | | | | |
| the invention 5 | | | | | |

^{a}Peak area A was the peak area at the peak position from 44 but less than 54 ppm
^{b}Peak area B was the peak area at the peak position from 54 to 58 ppm
^{c}Peak area C was the peak area at the peak position greater than 58 to 64 ppm
^{d}Peak area D was the peak area at the peak position from −10 to 5 ppm
^{e}Peak area ratio (A + C + D)/B was the total sum of peak areas A, C, and D divided by peak area B

TABLE 4
Efficacy of the catalyst in the cumene hydroperoxide decomposition using the
catalyst according to the invention compared to the comparative catalyst

| | Mole ratio of | Conversion of | | Conversion of | Yield of | Yield of |
|---|---|---|---|---|---|---|
| | silica to | cumene | Yield of | dimethyl | alpha- | para-cumyl |
| | alumina | hydroperoxide | phenol | phenylcarbinol | methyl | phenol |
| Sample | (SiO₂/Al₂O₃) | (%) | (%) | (%) | styrene (%) | (%) |
| Comparative | 30 | 91.4 | 91.2 | 65 | 46 | 2.8 |
| catalyst 3 | | | | | | |
| Comparative | 53 | 94.7 | 94.5 | 67 | 50 | 2.7 |
| catalyst 4 | | | | | | |
| Comparative | 233 | 100 | 99.8 | 36 | 20 | 2.4 |
| catalyst 5 | | | | | | |
| Comparative | 34 | 100 | 99.8 | 46 | 24 | 2.7 |
| catalyst 6 | | | | | | |
| Comparative | 37 | 100 | 99.8 | 71 | 52 | 3.0 |
| catalyst 7 | | | | | | |
| Comparative | 34 | 100 | 99.8 | 33 | 15 | 4.3 |
| catalyst 8 | | | | | | |
| Comparative | 52 | 100 | 99.8 | 43 | 24 | 2.6 |
| catalyst 9 | | | | | | |
| Comparative | 145 | 100 | 99.8 | 72 | 54 | 3.0 |
| catalyst 10 | | | | | | |
| Comparative | 180 | 100 | 99.8 | 68 | 51 | 3.1 |
| catalyst 11 | | | | | | |
| Catalyst | 53 | 100 | 99.9 | 83 | 68 | 1.4 |
| according to | | | | | | |
| the invention 1 | | | | | | |
| Catalyst | 59 | 100 | 99.9 | 96 | 87 | 1.5 |
| according to | | | | | | |
| the invention 2 | | | | | | |
| Catalyst | 74 | 100 | 99.9 | 96 | 86 | 1.5 |
| according to | | | | | | |
| the invention 3 | | | | | | |
| Catalyst | 61 | 100 | 99.8 | 97 | 87 | 1.7 |
| according to | | | | | | |
| the invention 4 | | | | | | |
| Catalyst | 71 | 100 | 99.9 | 98 | 88 | 1.4 |
| according to | | | | | | |
| the invention 5 | | | | | | |

From the analysis of the characteristics and structure of zeolite using x-ray diffraction (XRD) technique, it was found that the comparative catalyst 3 to 11 and the catalyst according to the invention 1 to 5 showed the characteristics of zeolite beta. This showed that the preparation process of the catalyst comprising the step of contacting zeolite beta with polycarboxylic acid solution and calcination at the temperature in the range from 400 to 700° C. still provided zeolite beta.

From table 3 which shows the peak area ratio obtained from the analysis using ²⁷Al magic angle spinning-nuclear magnetic resonance (²⁷Al MAS-NMR) technique according to the following relation: (A+C+D)/B, wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) was equal to 0.5 ppm, it was found that the catalyst according to the invention, in which the peak area ratio according to said relation was from 0.1 to 0.3, gave good efficacy for the cumene hydroperoxide decomposition as shown in table 4, which will be described as follows.

When comparing the catalyst sample according to the invention 1 to 5 with the comparative catalyst sample 1 to 9, it was found that the catalyst according to the invention gave good efficacy for the cumene hydroperoxide decomposition with complete conversion percentage of cumene hydroperoxide of 100%. Moreover, when considering the conversion percentage of dimethyl phenylcarbinol and yield percentage of alpha-methyl styrene, it was found that the catalyst sample according to the invention gave obviously higher value than the comparative catalyst sample and also gave lower yield percentage of para-cumyl phenol, which was the unwanted byproduct, than the comparative catalyst sample. Therefore, from the above experimental results, it could be said that the catalyst according to the invention had good efficacy for the cumene hydroperoxide decomposition as stated in the objective of this invention.

### BEST MODE OF THE INVENTION

Best mode or preferred embodiment of the invention is as provided in the description of the invention.

1. A catalyst for cumene hydroperoxide decomposition comprises zeolite beta, wherein said catalyst has the following characteristics:
a) the mole ratio of silica to alumina is from 30 to 100; and
b) the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

2. The catalyst according to claim 1, wherein the mole ratio of silica to alumina is from 50 to 100.

3. The catalyst according to claim 2, wherein the mole ratio of silica to alumina is from 50 to 80.

4. The catalyst according to claim 1, wherein (A+C+D)/B is from 0.1 to 0.25.

5. The catalyst according to claim 1, wherein said catalyst is prepared from the process comprising contacting zeolite beta with polycarboxylic acid solution and calcination at the temperature in the range from 400 to 700° C.;
wherein said polycarboxylic acid has the pKa of the first dissociation from 1.5 to 3.5 and has the molecular structure with at least one side having size larger than 6 Å.

6. The catalyst according to claim 5, wherein zeolite beta prior to contacting with polycarboxylic acid solution has the mole ratio of silica to alumina in the range from 20 to 50.

7. The catalyst according to claim 5, wherein said polycarboxylic acid has the pKa of the first dissociation in the range from 2.5 to 3.0.

8. The catalyst according to claim 5, wherein said polycarboxylic acid has the molecular structure with at least one side having size larger than 6 Å but not more than 30 Å.

9. The catalyst according to claim 5, wherein said polycarboxylic acid is selected from tartaric acid, citric acid, isocitric acid, aconitric acid, citraconic acid, 1,2,3,4-butanetetracarboxylic acid, ethylenediaminetetraacetic acid, nitriloacetic acid, diethylenetriaminepentaacetic acid (pentetic acid), hydroxyethylethylenediaminetriacetic acid, ethylenediaminedisuccinic acid, iminodiacetic acid, iminodisuccinic acid, methylglycinediacetic acid, 2-butenedioic acid, 1,1′-[[(3-carboxy-1-oxo-2-propen-1-yl)imino]di-2,1-ethanediyl] ester (DHEA), or mixture thereof.

10. The catalyst according to claim 5, wherein the weight ratio of zeolite beta to polycarboxylic acid solution is in the range from 1:10 to 1:30.

11. The catalyst according to claim 5, wherein the ratio of polycarboxylic acid to zeolite beta is in the range from 1 to 6 moles of polycarboxylic acid per kg of zeolite beta.

12. A preparation process of the catalyst for cumene hydroperoxide decomposition comprises the step of contacting zeolite beta with polycarboxylic acid solution and calcination at the temperature in the range from 400 to 700° C.;
wherein said polycarboxylic acid has the pKa of the first dissociation from 1.5 to 3.5 and has the molecular structure with at least one side having size larger than 6 Å.

13. The preparation process of the catalyst according to claim 12, wherein said catalyst has the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

14. The preparation process of the catalyst according to claim 13, wherein (A+C+D)/B is from 0.1 to 0.25.

15. The preparation process of the catalyst according to claim 12, wherein zeolite beta prior to contacting with polycarboxylic acid solution has the mole ratio of silica to alumina in the range from 20 to 50.

16. The preparation process of the catalyst according to claim 12, wherein said polycarboxylic acid has the pKa of the first dissociation from 2.5 to 3.0.

17. The preparation process of the catalyst according to claim 12, wherein said polycarboxylic acid has the molecular structure with at least one side having size larger than 6 Å but not more than 30 Å.

18. The preparation process of the catalyst according to claim 12, wherein said polycarboxylic acid is selected from tartaric acid, citric acid, isocitric acid, aconitric acid, citraconic acid, 1,2,3,4-butanetetracarboxylic acid, ethylenediaminetetraacetic acid, nitriloacetic acid, diethylenetriaminepentaacetic acid (pentetic acid), hydroxyethylethylenediaminetriacetic acid, ethylenediaminedisuccinic acid, iminodiacetic acid, iminodisuccinic acid, methylglycinediacetic acid, 2-butenedioic acid, 1,1′-[[(3-carboxy-1-oxo-2-propen-1-yl)imino]di-2,1-ethanediyl] ester (DHEA), or mixture thereof.

19. The preparation process of the catalyst according to claim 12, wherein the weight ratio of zeolite beta to polycarboxylic acid solution is in the range from 1:10 to 1:30.

20. The preparation process of the catalyst according to claim 12, wherein the ratio of polycarboxylic acid to zeolite beta is in the range from 1 to 6 moles of polycarboxylic acid per kg of zeolite beta.

21. The preparation process of the catalyst according to claim 12, wherein contacting zeolite beta with polycarboxylic acid solution is performed at the temperature in the range from 50 to 90° C.

22. The preparation process of the catalyst according to claim 12, wherein said process further comprises the step of washing with solvent and drying.

23. The preparation process of the catalyst according to claim 12, wherein the calcination is performed at the temperature in the range from 500 to 600° C.

24. A process for cumene hydroperoxide decomposition comprises the contact of the feed line comprising cumene hydroperoxide to the catalyst comprising zeolite beta in the reactor, and the separation of phenol and acetone from the product line, wherein said catalyst has the following characteristics:
a) the mole ratio of silica to alumina is from 30 to 100; and
b) the peak area ratio obtained from the analysis using ²⁷Al magic angle spinning-nuclear magnetic resonance (²⁷Al MAS-NMR) technique according to the following relation: (A+C+D)/B is from 0.1 to 0.3;
wherein A, B, C, and D are the peak area at the peak position from 44 but less than 54 ppm, the peak area at the peak position from 54 to 58 ppm, the peak area at the peak position greater than 58 to 64 ppm, and the peak area at the peak position from −10 to 5 ppm, respectively, by the peak deconvolution analysis from the ²⁷Al magic angle spinning-nuclear magnetic resonance technique with Lorentz/Gauss function under the condition that the minimum distance between peaks for independent integration (AZFW) is equal to 0.5 ppm.

25. The process according to claim 24, wherein the mole ratio of silica to alumina is from 50 to 100.

26. The process according to claim 25, wherein the mole ratio of silica to alumina is from 50 to 80.

27. The process according to claim 24, wherein (A+C+D)/B is from 0.1 to 0.25.

28. The process according to claim 24, wherein said catalyst is prepared from the process comprising contacting zeolite beta with polycarboxylic acid solution and calcination at the temperature in the range from 400 to 700° C.;
wherein said polycarboxylic acid has the pKa of the first dissociation from 1.5 to 3.5 and has the molecular structure with at least one side having size larger than 6 Å.

29. The process according to claim 28, wherein said polycarboxylic acid has the pKa of the first dissociation in the range from 2.5 to 3.0.

30. The process according to claim 28, wherein said polycarboxylic acid has the molecular structure with at least one side having size larger than 6 Å but not more than 30 Å.

31. The process according to claim 28, wherein said polycarboxylic acid is selected from tartaric acid, citric acid, isocitric acid, aconitric acid, citraconic acid, 1,2,3,4-butanetetracarboxylic acid, ethylenediaminetetraacetic acid, nitriloacetic acid, diethylenetriaminepentaacetic acid (pentetic acid), hydroxyethylethylenediaminetriacetic acid, ethylenediaminedisuccinic acid, iminodiacetic acid, iminodisuccinic acid, methylglycinediacetic acid, 2-butenedioic acid, 1,1′-[[(3-carboxy-1-oxo-2-propen-1-yl)imino]di-2,1-ethanediyl] ester (DHEA), or mixture thereof.

32. The process according to claim 28, wherein the weight ratio of zeolite beta to polycarboxylic acid solution is in the range from 1:10 to 1:30.

33. The process according to claim 28, wherein the ratio of polycarboxylic acid to zeolite beta is in the range from 1 to 6 moles of polycarboxylic acid per kg of zeolite beta.

34. The process according to claim 24, wherein the contact of the feed line to the catalyst is performed at the temperature in the range from 50 to 100° C.

35. The process according to claim 24, wherein the feed line further comprises dimethyl phenylcarbinol, and said process further comprises the dehydration of dimethyl phenylcarbinol.
